(19) United States
(12) Patent Application Publication
(10) Pub. No.: US 2003/0045793 A1
(43) Pub. Date: Mar. 6, 2003

(54) Device for forming diagnostic images of a patient

(76) Inventors: Peter Maria Johannes Rongen, Eindhoven (NL); Jacobus Johannes Maria Lukassen, Eindhoven (NL)

Correspondence Address: EUGENE E. CLAIR, Philips Medical Systems (Cleveland), Inc., 595 Miner Road, Cleveland, OH, 44143, US

(21) Appl. No.: 10/215,713

(22) Filed: Aug. 9, 2002

(30) Foreign Application Priority Data
Aug. 9, 2001 (EP) 01203029.2

(51) Int. Cl. A61B 5/05

(52) U.S. Cl. 600/425

(57) ABSTRACT

The invention relates to a device for forming diagnostic images of a patient, which device includes an excitation member for irradiation or exposure of the patient, a detector for receiving radiation and for converting the radiation into image information, an image processing member which is connected to the detector and has a functionality for enhancing or modifying the image information, a monitor which is connected to the image processing member, and a storage medium for the temporary storage of a series of images for a predetermined duration; during operation the image processing member executes a first sub-operation on the image information in order to provide pre-processed image information which is stored in the temporary storage medium, whereas during the display of the preprocessed image information from the temporary storage medium the image processing member executes at least a second sub-operation thereon, the first sub-operation and the second sub-operation together providing the functionality of the image processing member.

### BACKGROUND

The invention relates to a device for forming diagnostic images of a patient, which device includes an excitation member for irradiation or exposure of the patient, a detector for receiving radiation and for converting the radiation into image information, an image processing member which is connected to the detector and has a functionality for enhancing or modifying the image information, an image monitor which is connected to the image processing member, and a storage medium for the temporary storage of a series of images for a predetermined duration.

A device of this kind is known from practice. The known device includes, for example, an X-ray source or is based on the use of ultrasound for exposure of the patient. It is to be understood that hereinafter the invention will be described with reference to a device for diagnostic imaging by means of X-rays. However, it will be apparent that the invention is not restricted to such a type of device but can also be implemented in devices for computed tomography and devices based on magnetic resonance and ultrasound.

In a device of the kind set forth which is based on X-rays, images of the patient are continuously acquired during given examinations, implying that small X-ray doses should be used. This is done, for example, in cardio-angioplastiography. Because of the use of small X-ray doses, the images thus obtained have a poor signal-to-noise ratio, so that image details are lost. In practice it is desirable to process the acquired images so as to make such details visible as yet or, in a general sense, so as to enhance image aspects.

Because the acquired images become available in real time and comprise a large number of pixels, the desired image processing functionality necessitates a vast calculation capacity of the processor in the image processing member used for this purpose. The vast calculation capacity required usually impedes real-time execution.

Operation of the known device involves situations where the user wishes to review given, recently acquired images repeatedly at the actual speed for a closer study of given details. To this end, there is provided a storage medium which forms part of the device and serves for storing a series of images of a duration which is predetermined and limited. In exactly these circumstances it is desirable that the acquired images have been enhanced or modified by means of the image processing member, thus facilitating and supporting the analysis of the images. The problem as described above is then encountered in practice; in other words, the image processing member is not fast enough to execute the desired functionality without compromising the display rate of the images.

### SUMMARY OF THE INVENTION

It is an object of the invention to provide a device in which this problem is solved. To this end, the device in accordance with the invention is characterized in that during operation the image processing member executes a first sub-operation on the image information in order to provide pre-processed image information, that the temporary storage medium stores said pre-processed image information, and that upon display of the preprocessed image information from the temporary storage medium the image processing member executes at least a second sub-operation thereon, the first sub-operation and the second sub-operation together providing the functionality of the image processing member.

It is to be noted that a device involving multiple signal processing for the formation of diagnostic images of a patient is already known from EP-A-0 149 516. However, the known device involves parallel-operating image processors which execute functions which are distinct from one another and each of which can be executed in real time. The system as known from EP-A-0 149 516, however, does not offer a solution for the situation where the functionality to be realized by the image processing member requires such a vast calculation capacity that display of the processed images at the original image rate is not possible. Moreover, the system as known from EP-A-0 149 516 is comparatively complex and expensive because of the use of a plurality of processors and the associated control software.

In a first embodiment of the device in accordance with the invention in the first sub-operation each image is formed into the pre-processed image information by the image processing member, which pre-processed image information is subsequently stored in the storage medium after which it is subjected to the finishing, second sub-operation.

It is possible to execute the second sub-operation on each individual preprocessed image separately. In an advantageous further embodiment of the device in accordance with the invention, in which the pre-processed image information is repeatedly displayed from the temporary storage medium, however, the second image operation is performed alternately on a first image and a second image of every two successive images. This offers a further saving in respect of processor time; such a saving is advantageous notably when the second sub-operation is of a comparatively complex nature and requires a large processor capacity.

In a further embodiment of the device in accordance with the invention, prior to the storage in the storage medium the image processing member executes the first sub-operation on each time only a first one of every two successive images, the image processing member executing the second sub-operation on each time the second one of every two successive images upon display of the pre-processed image information from the storage medium. In this embodiment the first sub-operation and the second sub-operation have the same functionality and, consequently, the splitting of the functionality of the image processing member takes place in that the image processing member is alternately active on a first image and a second image, respectively, of every two successive images, the images being stored in the storage medium between the two sub-operations. This offers the advantage that it is still possible to display also non-processed image information from the storage medium.

It may be advantageous to display the pre-processed image information from the storage medium in a delayed fashion. This enables the application of very complex image processing operations.

The following description, claims and accompanying drawings set forth certain illustrative embodiments applying various principles of the present invention. It is to be appreciated that different embodiments applying principles of the invention may take form in various components, steps and arrangements of components and steps. These described embodiments being indicative of but a few of the various ways in which some or all of the principles of the invention may be employed in a method or apparatus. The drawings are only for the purpose of illustrating an embodiment of an apparatus and method applying principles of the present invention and are not to be construed as limiting the present invention.

### BRIEF DESCRIPTION OF THE DRAWINGS

The foregoing and other features and advantages of the present invention will become apparent to those skilled in the art to which the present invention relates upon consideration of the following detailed description of apparatus applying aspects of the present invention with reference to the accompanying drawings, wherein:

FIG. 1 is a diagrammatic representation of the device in accordance with the invention;

FIG. 2A is a diagrammatic representation of the operation of the image processing member forming part of the device shown in FIG. 1; and

FIG. 2B is a diagrammatic representation of the operation of the image processing member forming part of the device shown in FIG. 1.

### DETAILED DESCRIPTION

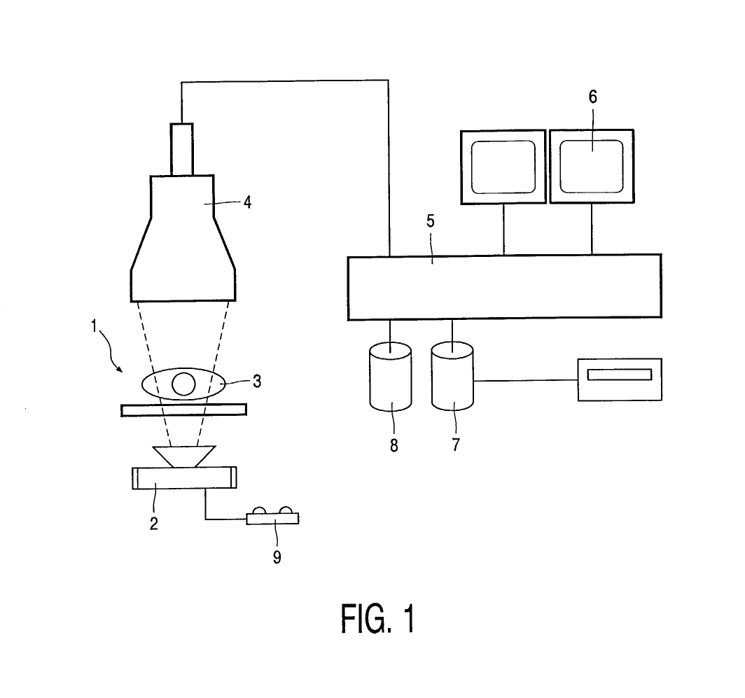

Throughout the following description, corresponding reference numerals in the Figures denote corresponding components. FIG. 1 shows the device 1 for the formation of diagnostic images of the patient. The present embodiment of the device 1 concerns an examination device on the basis of X-rays. As has already been noted, however, the invention is not restricted to this type of device and may also concern a device which is based on ultrasound or magnetic resonance. The invention can also be used in a computed tomography device.

The device 1 as shown in Fig. i includes an excitation member 2 in the form of an X-ray source which serves to irradiate a patient 3. The device 1 also includes a detector 4 for receiving the radiation having traversed the patient 3 and for converting the radiation into image information which is to be further processed in an image processing member 5 which is connected to the detector 4. At least one monitor 6 for the display of images that have been processed or not is connected to the image processing member 5. A storage medium 7 for storing the detected images is also connected to the image processing member 5. The device 1 in accordance with the invention is also provided with a storage medium 8 for the temporary storage of a series of images for a predetermined duration. This temporary storage medium 8 may be considered to be a buffer for the last images acquired by means of the detector 4. The temporary storage medium 8 enables the user of the device to perform a closer study (by operating a switch 9 which is usually a foot switch) of the images stored on the temporary storage medium 8, that is, via the monitor 6. The image processing member 5 serves to optimize the images displayed via the monitor 6.

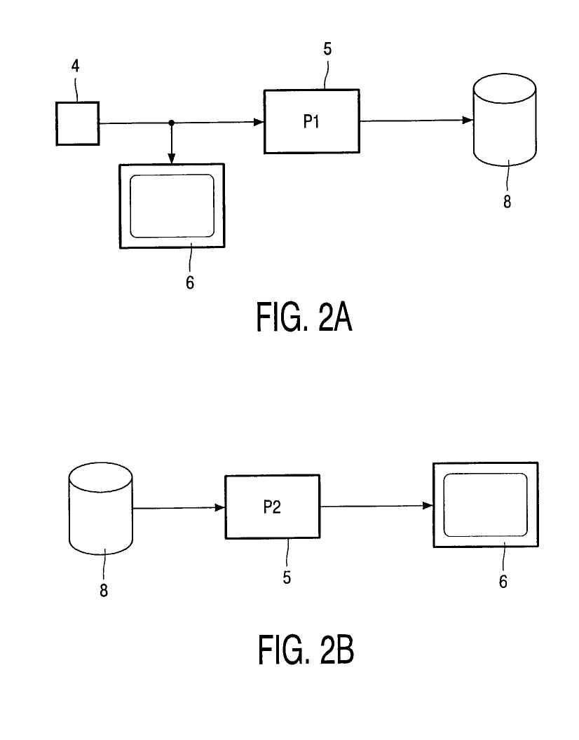

The section 2a of FIG. 2 shows that while the images of the detector 4 appear on the monitor 6 in real time, the image processing member 5 executes a first sub-operation PI in order to produce pre-processed image information which is subsequently stored in the temporary storage medium 8. FIG. 2b shows that, upon display of the image information from the temporary storage medium 8, the image processing member 5 performs at least a second sub-operation P2 thereon. The first sub-operation P1 shown in FIG. 2a and the second sub-operation P2 shown in FIG. 2b together define the functionality of the image processing member 5 such that, after execution of the second sub-operation P2, the enhanced or modified image information can be displayed at the original image rate on the monitor 6.

As an alternative version to the described splitting of the functionality of the image processing member 5 it is also possible to execute the second sub-operation P2 during repeated display of the pre-processed image information from the temporary storage medium 8, that is, alternately on a first image and a second image, respectively, of every two successive images. As a result, the second sub-operation P2 may be of a comparatively complex nature so that it requires a comparatively large amount of processor time.

It is to be noted that it is also possible to perform the first sub-operation P1 and the second sub-operation P2 in an identical fashion, said sub-operations being realized in such a way that the first sub-operation P1 is executed, prior to the storage of the image information on the storage medium 8, on each time a first image of every two successive images. The image processing member 5 in that case subsequently executes the second sub-operation P2 on each time only the second image of every two successive images upon display from the storage medium 8. In that case at least half of the images stored in the temporary storage medium 8 remain unprocessed, so that they remain available, for example, for reference purposes.

It is also to be noted that the display of the pre-processed image information from the temporary storage medium 8 may also be carried out in a delayed fashion in order to create room for very calculate-intensive sub-operations P2 to be carried out by the image processing member 5.

The invention is of course not limited to the described or shown embodiments, but generally extends to any embodiment, which falls within the scope of the appended claims as seen in light of the foregoing description and drawings. While a particular feature of the invention may have been described above with respect to only one of the illustrated embodiments, such features may be combined with one or more other features of other embodiments, as may be desired and advantageous for any given particular application. From the above description of the invention, those skilled in the art will perceive improvements, changes and modification. Such improvements, changes and modification within the skill of the art are intended to be covered by the appended claims.

Having described a preferred embodiment of the invention, the following is claimed:

1. A device for forming diagnostic images of a patient, the device comprising an excitation member for irradiation of the patient, a detector for receiving radiation and for converting the radiation into image information, an image processing member connected to the detector, the image processing member adapted for at least one of enhancing and modifying the image information, an image monitor connected to the image processing member, and a storage medium for the temporary storage of a series of images for a predetermined duration, wherein the image processing member includes means for executing a first sub-operation on the image information to provide pre-processed image information, and the image processing member includes means for executing at least a second sub-operation, the first sub-operation and the second sub-operation together providing the functionality of the image processing member.

2. The device of claim 1 wherein during repeated display of the pre-processed image information from the temporary storage medium the image processing member executes the second sub-operation alternately on a first image and a second image, respectively, of every two successive images.

3. The device of claim 1 wherein the image processing member executes the first sub-operation on each time only a first image of every two successive images, and the image processing member executes the second sub-operation on each time the second image of every two successive images.

4. The device of claim 1, wherein the pre-processed information from the storage medium is displayed in a delayed fashion.

5. A device for forming diagnostic images of a patient, the device comprising:
an excitation member for irradiation or exposure of the patient;
a detector for receiving radiation and for converting the radiation into image information;
an image processing member connected to the detector, the image processing member adapted for at least one of enhancing and modifying the image information, the image processing member adapted to execute a first sub-operation on the image information in order to provide pre-processed image information, the image processing member adapted to execute at least a second sub-operation;
an image monitor connected to the image processing member; and
a storage medium for the temporary storage of a series of images for a predetermined duration, wherein during operation the image processing member executes the first sub-operation on the image information in order to provide pre-processed image information, the temporary storage medium in data communication with the first sub-operation to store the pre-processed image information, and upon display of the preprocessed image information on the image monitor from the temporary storage medium the image processing member executes at least the second sub-operation thereon.

6. The device of claim 5, wherein during repeated display of the pre-processed image information from the temporary storage medium the image processing member is adapted to execute the second sub-operation alternately on a first image and a second image, respectively, of every two successive images.

7. The device of claim 5, wherein the image processing member is adapted to execute the first sub-operation on each time only a first image of every two successive images, and that the image processing member executes the second sub-operation on each time the second image of every two successive images.

8. The device of claim 5, wherein the pre-processed information from the storage medium is displayed in a delayed fashion.

9. A device for forming diagnostic images of a patient, the device comprising:
an excitation member for irradiation or exposure of the patient;
a detector for receiving radiation and for converting the radiation into image information;
an image processing member connected to the detector, the image processing member adapted for at least one of enhancing and modifying the image information;
an image monitor connected to the image processing member; and
a storage medium for the temporary storage of a series of images for a predetermined duration, wherein the image processing member includes means for executing a first sub-operation on the image information in order to provide pre-processed image information, and the image processing member includes means for executing at least a second sub-operation of the pre-processed image information from the temporary storage medium.

10. The device of claim 9, wherein during repeated display of the pre-processed image information from the temporary storage medium the image processing member is adapted to execute the second sub-operation alternately on a first image and a second image, respectively, of every two successive images.

11. The device of claim 9, wherein that the image processing member is adapted to execute the first sub-operation on a first set of alternating images of every two successive images, and that the image processing member is adapted to execute the second sub-operation on the other alternate set of every two successive images.

12. The device of claim 9, wherein the pre-processed information from the storage medium is displayed in a delayed fashion.

13. A method for forming diagnostic images of a patient, the method comprising
irradiating a patient;
detecting radiation;
converting the radiation into image information;
storing a series of images for a predetermined duration;
processing the image information for at least one of enhancing and modifying the image information, wherein the step of image processing includes executing a first sub-operation on the image information to provide pre-processed image information and temporarily storing the pre-processed image information; and
displaying of the pre-processed image information from the temporary storage medium on an image monitor, the step of image processing including executing at least a second sub-operation thereon.

14. The method of claim 13 wherein during repeated display of the pre-processed image information from the temporary storage medium the step of image processing includes execution the second sub-operation alternately on a first image and a second image, respectively, of every two successive images.

15. The method of claim 13, wherein the step of image processing includes execution of the first sub-operation on each time only a first image of every two successive images, and that the step of image processing includes the step of executing the second sub-operation on each time the second image of every two successive images.

16. The method of claim 13 wherein the pre-processed information from the storage medium is displayed in a delayed fashion.
